(19) United States
(12) Patent Application Publication
(10) Pub. No.: US 2026/0306696 A1
(43) Pub. Date: Oct. 8, 2026

(54) DISINFECTION DEVICES FOR EYE CARE ACCESSORIES AND METHODS OF USE

(71) Applicant: Drop Mate, Inc., Sunnyvale, CA (US)

(72) Inventors: Vikram Shankar, Sunnyvale, CA (US); Phillip Ianchulev, Sunnyvale, CA (US); Alexander Ianchulev, Sunnyvale, CA (US)

(21) Appl. No.: 19/484,772

(22) PCT Filed: May 15, 2024
(86) PCT No.: PCT/US2024/029419

§ 371 Date: Nov. 14, 2025

Related U.S. Application Data
(60) Provisional application No. 63/502,899, filed on May 17, 2023.
(60) Provisional application No. 63/600,423, filed on Nov. 17, 2023.

(51) Int. Cl. A61L 12/06; A45C 11/00; A45C 15/06

(52) U.S. Cl. CPC A61L 12/063; A45C 11/005; A45C 15/06; A45C 2011/007

(57) ABSTRACT

A treatment device configured for storing and disinfecting one or more eye care accessories comprising a housing comprising at least one treatment bay sized to accommodate and support an eye care accessory; and a plurality of ultraviolet light sources disposed within the housing, the plurality of ultraviolet light sources having an orientation, number, and radiant flux sufficient for achieving disinfection of the eye care accessory within the treatment bay. Related devices, systems, and methods are provided.

### CROSS-REFERENCE TO RELATED APPLICATIONS

This application claims the benefit of priority under 35 U.S.C. § 119(e) to U.S. Provisional Application Ser. No. 63/502,899, filed May 17, 2023, and 63/600,423, filed Nov. 17, 2023. The disclosures are each incorporated by reference in their entireties.

### BACKGROUND

Ophthalmic medical devices, medication solutions, and drops are used by hundreds of millions of patients worldwide to manage eye problems including dry eye, allergies, glaucoma, inflammation, and other chronic eye diseases. Eyedropper bottles are manufactured, filled, and sold in the sterile form, but neither the interior nor exterior of the cap, nozzle, or bottle remain sterile once the seal is broken by the user.

Over weeks or months of repeated use, eyedropper bottles may become contaminated with microorganisms, putting millions of patients with chronic eye diseases at risk for serious and blinding infection and vision loss (Nentwich et al. Br. J. Ophthalmol. (2007) 91(10):1265-8). A plethora of studies indicate that up to 70% of eyedroppers grow bacteria from the surface of the nozzle or from the interior after only a few days of use (Teuchner et al. Medicine (United States). (2015). 94(8): e538). This risk is further increased by the duration of use, certain designs of the eyedropper tip, and improper application technique (da Costa et al. Transl Vis Sci Technol. 2020; 9(7):1-7).

Some ophthalmic medications are formulated without preservatives to limit repeated patient exposure to compounds that can irritate the ocular surface. Eye dropper contamination is particularly problematic for these preservative-free formulations, as pathogenic growth within the solution is unimpaired. Eye dropper contamination by various fungi and/or bacteria can result in severe infection, blindness, loss of an eye, and in some extreme cases death.

In medical settings, the risk for cross-contamination between patients with multi-use devices or bottles is significant, as they may be handled by multiple users and shared among dozens or more patients over days to weeks. Physicians will often wipe ophthalmic medical devices such as applanation tonometer prism tips or rebound tonometers with isopropyl alcohol swabs between patients, which provides insufficient virucidal or sporicidal effect. Alcohol swabs cannot safely be used on eyedropper nozzles, as residual solution can be toxic to the corneal epithelium if delivered along with the intended medication. Preservative-free drops are often packaged into single-use vials that cannot be closed and are not intended to be used repeatedly to reduce the risk of contamination. However, this packaging is wasteful, and depending on the end-user may also be used multiple times against manufacturer instructions.

In view of the foregoing, there is a need for improved sanitization and disinfection devices for eye care accessories.

### SUMMARY

In an aspect, provided is a treatment device configured for storing and disinfecting one or more eye care accessories. The treatment device includes a housing comprising at least one treatment bay sized to accommodate and support an eye care accessory; and a plurality of ultraviolet light sources disposed within the housing, the plurality of ultraviolet light sources having an orientation, number, and radiant flux sufficient for achieving disinfection of the eye care accessory within the treatment bay.

The eye care accessory can include a surface designed to contact an anterior surface of a patient's eye directly or indirectly. The eye care accessory can be an eyedropper bottle, eye dropper bottle cap, contact lens, contact lens case, applanation tonometer prism tip, rebound tonometer probe, ophthalmic prism lens, gonioprism, condensing lens, or other contact ophthalmic lens. The housing can include a base and a covering movably coupled to the base, the covering having an open configuration allowing access to the treatment bay of the housing and a closed configuration. The treatment bay can include a lower surface near a lower end region of the treatment bay, a ceiling near an upper end region that is positioned over at least a portion of the lower surface, and at least one side wall. A first light source of the plurality of ultraviolet light sources can be arranged within a first bank located within the at least one side wall. The first bank can include the first light source and a second light source. The first light source can be near the upper end region of the treatment bay and the second light source can be near the lower end region of the treatment bay. At least one light source of the plurality of ultraviolet light sources can be located within the ceiling of the treatment bay.

The at least one side wall can form a geometric cross-sectional shape to the treatment bay. The at least one side wall can include one side wall and the geometric cross-sectional shape of the treatment bay is a circle or oval. The at least one side wall can include four side walls and the geometric cross-sectional shape of the treatment bay can be a square or rectangle. Each of the four side walls can include a bank of light sources of the plurality of ultraviolet light sources. The housing can include three treatment bays, each treatment bay sized to accommodate and support one eye care accessory. Each bay of the three treatment bays can include banks of light sources of the plurality of light sources, each bank of light sources arranged to create multiple overlapping cones of ultraviolet light.

Each light source of the plurality of ultraviolet light sources can be a UV-C light source. The UV-C light source can be a UV-C light emitting diode (LED). The UV-C LED can be powered to achieve high-level disinfection of the eye care accessory within an exposure period, high-level disinfection causing 6 log reduction in bacterial concentration and 4 log reduction in viral concentration. The exposure period can be 1 minute to 5 minutes. The exposure period can be cycled at least one cycle per day up to 100 cycles per day. The UV-C LED has a threshold energy delivery that is at least 160 mJ/cm². A first light source of the plurality of ultraviolet light sources can be located within the ceiling of the treatment bay and a second light source of the plurality of ultraviolet light sources can be located within the at least one side wall. A radiant flux of the first light source can be greater than a radiant flux of the second light source. The second light source can be located within the at least one side wall and include an upper light emitting diode (LED) separated vertically from a lower light emitting diode (LED). The upper LED can create an upper cone of light and the lower LED can create a lower cone of light. The upper cone of light and lower cone of light can overlap.

A distance between the lower surface and the ceiling can be designed to ensure that, when the eye care accessory is positioned on the lower surface, a target surface of the eye care accessory is located within a threshold treatment zone of the first light source. A distance between the lower surface and the second light source can be designed to ensure that, when the eye care accessory is positioned on the lower surface, the target surface of the eye care accessory is located within at least one of the upper cone of light and the lower cone of light. The threshold treatment zone of the first light source can be at least 7 mm and no greater than about 60 mm from the first light source. The threshold treatment zone of the first light source can be at least 15 mm and no greater than 60 mm away from the first light source. The threshold treatment zone of the first light source can be at least 15 mm and no greater than 40 mm away from the first light source. The at least one of the upper cone of light and the lower cone of light can be an 80-degree cone that directly exposes the target surface to a threshold energy delivery that is at least 160 mJ/cm².

The treatment device can further include a microprocessor and a power system. The microprocessor can be programmed to provide timing of disinfection. The microprocessor can be a wireless circuitry. The wireless circuitry can include Wi-Fi, Zigbee, Bluetooth, or Internet of Things (IoT) connectivity.

In an interrelated aspect, provided is a method of storing and disinfecting one or more eye care accessories including enclosing an eye care accessory within a treatment bay of a treatment device; sealing the treatment device; applying ultraviolet radiation to the eye care accessory to achieve a total energy delivery that ensures disinfection within an exposure period; and preventing escape of the ultraviolet radiation from the treatment device while applying the ultraviolet radiation to the eye care accessory.

The eye care accessory can include a surface designed to contact an anterior surface of a patient's eye directly or indirectly. The eye care accessory can be an eyedropper bottle, eye dropper bottle cap, contact lens, contact lens case, applanation tonometer prism tip, rebound tonometer probe, ophthalmic prism, gonioprism, condensing lens, or ophthalmic lens. Enclosing the eye care accessory within the treatment bay can include positioning the eye care accessory on a lower surface near a lower end region of the treatment bay under a ceiling near an upper end region and adjacent at least one side wall. A first ultraviolet light source can be located within the ceiling of the treatment bay and a second ultraviolet light source can be located within the at least one side wall. A radiant flux of the first ultraviolet light source can be greater than a radiant flux of the second ultraviolet light source. The second ultraviolet light source located within the at least one side wall can be an upper light emitting diode (LED) separated vertically from a lower light emitting diode (LED). The upper LED can create an upper cone of light and the lower LED can create a lower cone of light. The upper cone of light and lower cone of light can overlap. A distance between the lower surface and the ceiling can be designed to ensure that, when the eye care accessory is positioned on the lower surface, a target surface of the eye care accessory is located within a threshold treatment zone of the first light source. A distance between the lower surface and the second light source can be designed to ensure that, when the eye care accessory is positioned on the lower surface, the target surface of the eye care accessory is located within at least one of the upper cone of light and the lower cone of light.

The threshold treatment zone of the first light source can be at least 15 mm and no greater than 60 mm away from the first light source. The at least one of the upper cone of light and the lower cone of light can be an 80-degree cone that exposes the target surface to a threshold energy delivery that is at least 160 mJ/cm². The treatment device can include three treatment bays, each treatment bay being sized to accommodate and support one eye care accessory. Applying ultraviolet radiation can include activating a plurality of UV-C light emitting diodes (LEDs) powered to achieve high-level disinfection of the eye care accessory within the exposure period, high-level disinfection causing 6 log reduction in bacterial concentration and 4 log reduction in viral concentration. The exposure period can be 1 minute to 5 minutes. The method can further include cycling the exposure period at least one cycle per day up to 100 cycles per day. The UV-C LEDs can have a threshold energy delivery that is at least 160 mJ/cm². The method can further include cleaning the eye care accessory with a wipe prior to enclosing within the treatment bay.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

These and other aspects will now be described in detail with reference to the following drawings. Generally, the figures are not to scale in absolute terms or comparatively but are intended to be illustrative. Also, relative placement of features and elements may be modified for the purpose of illustrative clarity.

FIG. 1A is a box diagram of an implementation of a device for sanitizing and disinfecting one or more eye care accessories;

FIG. 1B is a box diagram of a treatment system of the device of FIG. 1A;

FIG. 1C is a box diagram of a containing system of the device of FIG. 1A;

FIG. 2A is a perspective view of an implementation of a housing of the device of FIG. 1A;

FIG. 2B is a front view of the device of FIG. 2A;

FIG. 2C is a perspective rear view of the device of FIG. 2A in a closed configuration;

FIG. 2D is a perspective front side view of the device of FIG. 2A in an open configuration;

FIG. 2E is a perspective front side view of the device of FIG. 2D in the open configuration holding an eye care accessory having a first size;

FIG. 2F is a perspective front side view of the device of FIG. 2D in the open configuration holding an eye care accessory having a second, larger size;

FIG. 3 is a perspective view of an implementation of a housing of a device for sanitizing and disinfecting with the lid removed from the base;

FIG. 4 is a perspective view of another implementation of a housing of a device for sanitizing and disinfecting with the lid removed from the base;

FIG. 5 is a perspective view of an implementation of an adaptor for positioning within a housing of a device for sanitizing and disinfecting;

FIG. 6A is a perspective front side view of a device for sanitizing and disinfecting a plurality of eye care accessories;

FIG. 6B is a view of the device of FIG. 6A in an open configuration;

FIGS. 6C-6D are schematic views of the device of FIG. 6A showing relative position of the light sources within each treatment bay;

FIG. 7A is a perspective front side view of a device for sanitizing and disinfecting a plurality of eye care accessories in a closed configuration;

FIG. 7B is a perspective front side view of the device of FIG. 7A in an open configuration;

FIG. 7C is a perspective rear view of a device illustrating a power cable arrangement;

FIG. 8A is a partial side view of a device incorporating three treatment bays;

FIG. 8B is a perspective view of the treatment bays of FIG. 8A;

FIG. 8C is a schematic view of a treatment bay showing multiple overlapping cones of ultraviolet light relative to different sized accessories;

FIG. 8D is a schematic view of the treatment bay showing multiple overlapping cones of ultraviolet light for other accessories supported on a lifted lower surface;

FIGS. 9A-9B illustrate radiant flux of different power LEDs to illustrate usable range distances between light source and accessories of difference sizes;

FIG. 10A is a side view of a replacement cap positioned over a nozzle of an eye dropper bottle;

FIG. 10B is an exploded view of the replacement cap of FIG. 10A.

It should be appreciated that the drawings are for example only and are not meant to be to scale. It is to be understood that devices described herein may include features not necessarily depicted in each figure.

### DETAILED DESCRIPTION

Disclosed are point-of-care devices that provide rapid disinfection of eye care accessories for ophthalmology use, preferably accessories that come into contact with an anterior surface of an eye, directly and indirectly, such as in-use eye drop bottles, ophthalmic lenses, ophthalmic applanation prisms, gonioprisms, rebound tonometers, contact lens, and contact lens cases after initial sterility (provided by the fill-finish manufacturing processes) is broken. The treatment of the eye care accessory provided by the devices involves disinfection without heat, steam, chemical agents, abrasives, wipes, or the like and relies instead upon ultraviolet light to disinfect essential surfaces of the accessories within a short treatment cycle. The devices described herein store and protect the eye care accessories against heat, light, and contaminants while disinfecting using ultraviolet light. As will be discussed in more detail below, the device can be configured to be combined with smart monitoring functions such as patient compliance monitoring, reminder functions, audio and LED signaling, and other output(s).

Ultraviolet (“UV”) light or ultraviolet germicidal irradiation (UVGI) is a known disinfection method that employs UV light at sufficiently short wavelengths to kill microorganisms. UV light is used to control the growth of and bio-decontaminate microbial impurities in a variety of industries due to its germicidal effects. UV rays with a wavelength between 100 nm and 280 nm, particularly between 250 nm and 280 nm, effectively kill bacteria, viruses, yeast, molds, and algae. Without limiting this disclosure to any particular theory on the mechanism of action of ultraviolet germicidal irradiation, it is believed the UV radiation breaks through the outer membranes of such organisms, destroying or inactivating their DNA and preventing them from replicating. Eye care accessories typically have a contact surface that is a surface intended to contact a patient's eye for diagnostic or therapeutic purposes. Non-contact surfaces of an eye care accessory are not intended to come into contact with an eye, such as a lens handle or portion of the accessory that a user may hold during use of the accessory. The devices described herein provide in-field secondary disinfection with multiple ultraviolet (UV) light sources that emit wrap-around short wavelength UV-C light to disinfect at least one or more contact surfaces of eye care accessories, such as nozzle, threads, cap of an eyedropper bottle, the contact surface of an ophthalmic lens, applanation prism, tonometer probe, or others to prevent contamination. The devices described herein can also provide disinfection of one or more non-contact surfaces of eye care accessories. The devices described herein can provide germicidal illumination of the uncapped and/or capped surface of the nozzle and eyedropper bottle. The devices described herein are particularly suitable for personal use at home and/or on the go and for professional use within clinical setting, including eye clinics, hospitals, operating rooms, and the like.

The Centers for Disease Control and Prevention (CDC) provides guidelines for disinfection and sterilization (see, e.g., www.cdc.gov/infectioncontrol/guidelines/disinfection/index.html). “Sterilization” is generally used to describe a process that destroys or eliminates all forms of microbial life including bacterial endospores. “Disinfection” is generally used to describe a process that eliminates many or all pathogenic microorganisms on inanimate objects including viruses, but not necessarily all microbial forms (e.g., bacterial endospores). There are three levels of disinfection including high-, intermediate-, and low-level disinfection. High-level disinfection kills all organisms, except high levels of bacterial endospores. Intermediate-level disinfection kills mycobacteria, most viruses, and bacteria. Low-level disinfection kills some viruses and bacteria. “High-level disinfection” as used herein means disinfection that results in 6 log reduction in bacterial concentration and 4 log reduction in viral concentration within an exposure period that is less than about 3-12 hours, or within about 20 minutes, preferably less than about 10 minutes, less than about 5 minutes, less than about 3 minute, or about a 1 minute treatment cycle, or any time in between, including between 1 minute and 3 hours, or between 1 minute and 20 minutes, or between 1 minute and 10 minutes, or between 1 minute and 5 minutes, or between 1 minute and 3 minutes. “Sanitization” as used herein means a process that eliminates many or all pathogenic microorganisms on inanimate objects, except viruses and bacterial spores. The devices described herein are configured to accomplish disinfection of one or more contact surfaces of eye care accessories (i.e., surfaces of an accessory that are intended to contact an anterior surface of a patient's eye, directly or indirectly) using UV light. In several implementations, the devices are designed to achieve high-level disinfection using UV light. Where the devices are referred to herein as a sanitizing device it should be appreciated that the device is capable of disinfection, including low-level, intermediate-level, and/or high-level disinfection, or sterilization.

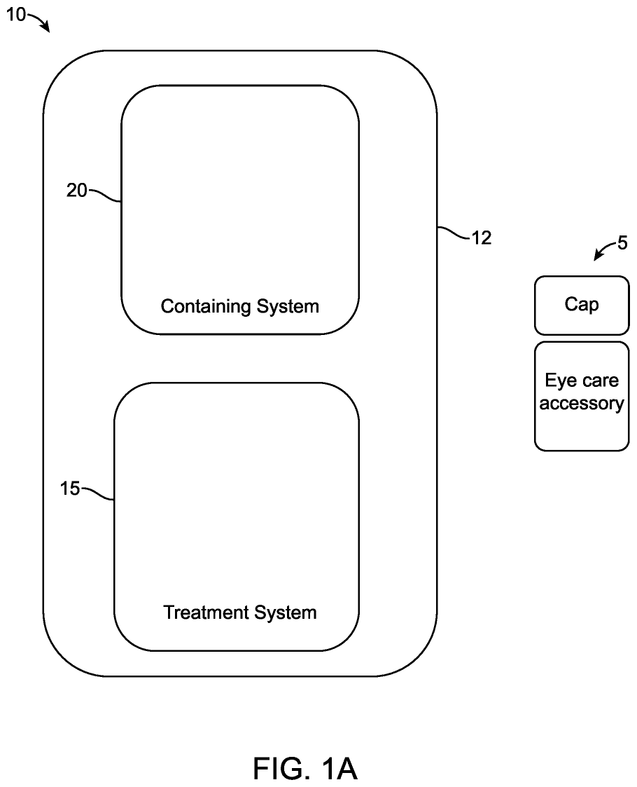

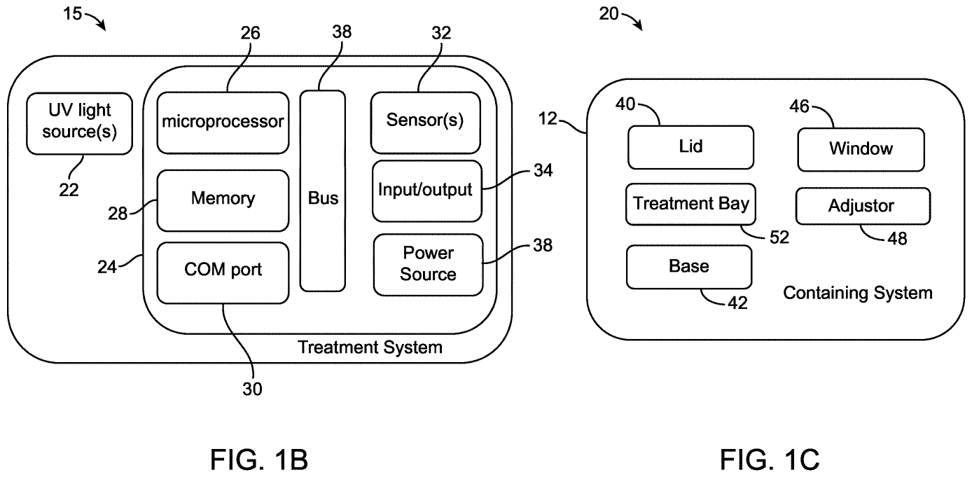

Turning now to the figures, FIG. 1A is a box diagram of an implementation of a treatment device 10 for disinfecting one or more eye care accessories 5 through direct exposure of contact surfaces of each of the accessories 5 to ultraviolet radiation. The device 10 can include a treatment system 15 and a containing system 20. The treatment system 15 provides ultraviolet light treatment of the one or more eye care accessories 5 held by the containing system 20. The containing system 20 can support the one or more eye care accessories 5 within a housing 12 for exposure to the ultraviolet light treatment.

FIG. 1B is a box diagram of an implementation of the treatment system 15 for the device 10 of FIG. 1A. The treatment system 15 can include at least one, preferably a plurality, of irradiating UV light sources 22 and a control module 24. The UV light sources 22 have an intensity sufficient for sanitization and disinfection of the one or more eye care accessories 5 enclosed within the interior cavity of the housing 12.

As will be discussed in more detail below, the UV light sources 22 can be UV-C light emitting diodes (LEDs) that provide focal, wrap-around, or diffuse illumination of each eye care accessory contained within the housing 12. The control module 24 can include a microprocessor 26, one or more input/outputs 34 ports, and a power system 36. The control module 24 may also include a memory 28, a COM port 30, one or more sensors 32, and the like. The various components of the control module 24 can be interconnected by a system bus 38. Each of these will be described in more detail below.

FIG. 1C is a box diagram of an implementation of the containing system 20 including a housing 12 formed by a lid 40 movably coupled to a base 42. The lid 40 and base 42 together define at least one interior cavity of the containing system 20 sized to receive and enclose the one or more eye care accessories 5. The housing 12 can include at least one treatment bay 52 including a lower surface 44 for supporting an eye care accessory 5 and at least one side wall 47 for surrounding at least a portion of the eye care accessory 5 located within the treatment bay 52. The containing system 20 can additionally incorporate a window 46 through at least a portion of the housing 12 for inspection of an interior of the housing 12 without risk of exposure to UV light. Optionally, the containing system 20 can incorporate an adjustor 48 configured to move a lower surface 44 of the treatment bay 52 (also referred to herein as a floor or stage) supporting the eye care accessory 5 within the housing 12 relative to the base 42. Each of these components will be described in more detail below.

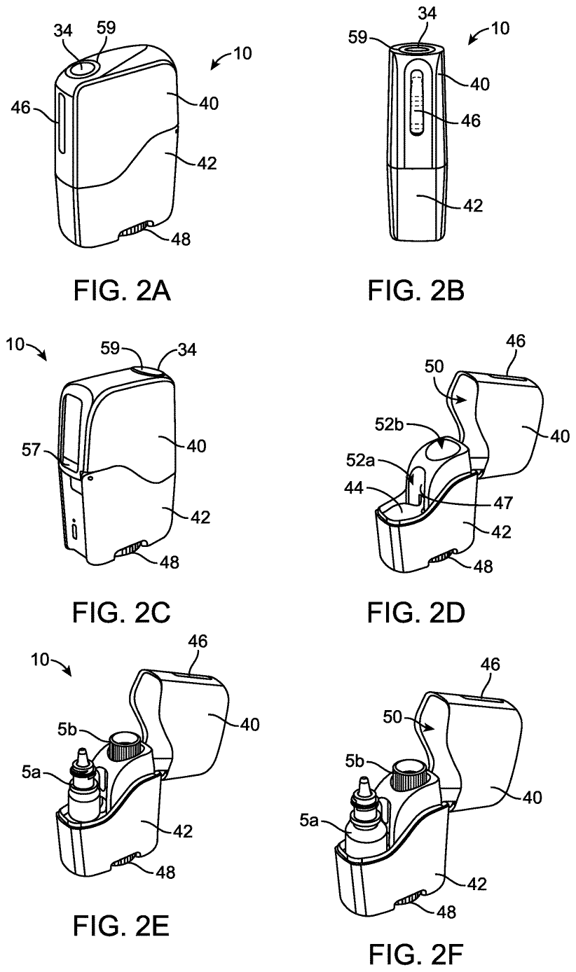

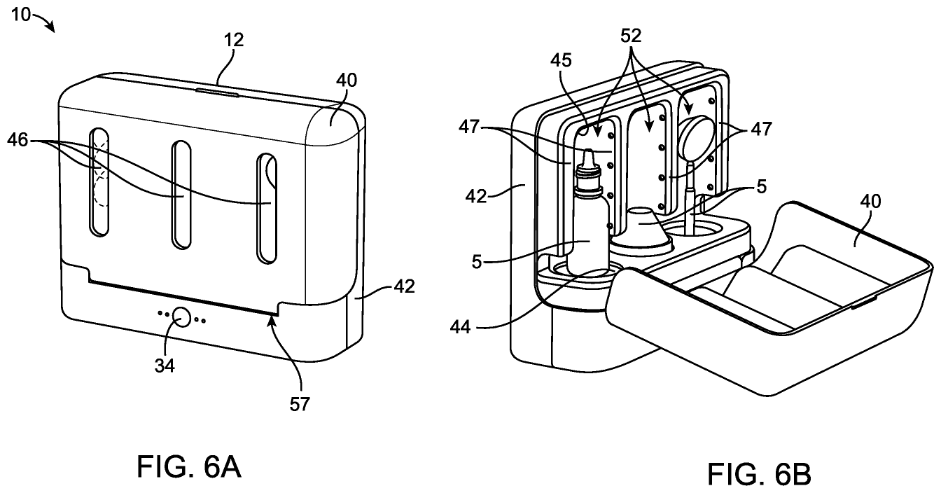

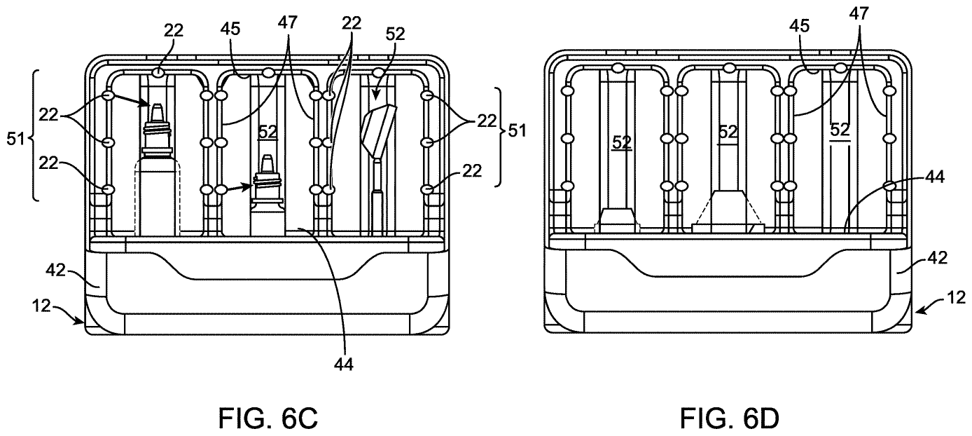

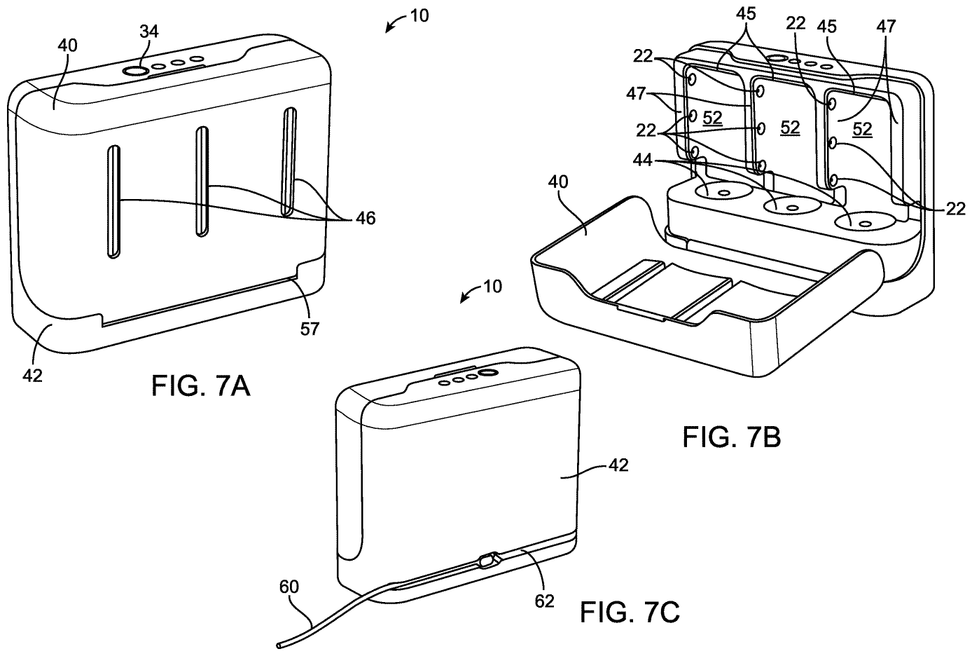

Preferably, the device 10 is sized to be portable. In some implementations, the portable device 10 has a form factor suitable for single-hand use and that is sized to be positioned within a pocket or purse (see FIGS. 2A-2F, 3, and 4). In other implementations, the portable device 10 has a form factor more suitable for positioning on a desktop or tabletop, such as a countertop in an ophthalmology or optometry office (see FIGS. 6A-6D, 7A-7C). The devices described herein can feature a multi-functional design that can be varied and modified to accommodate one or a plurality of reusable and/or disposable eye care accessories 5. The one or more eye care accessories 5 contained within the housing 12 of the device 10 (i.e., within the boundaries of the lid 40 and the base 42) can vary including one or more disposable and/or reusable eye care tools that come in direct contact with a patient's eye surface (e.g., anterior surface of the eyeball, including cornea, sclera, conjunctiva, etc.), such as applanation tonometer prism tips, rebound tonometer probes, ophthalmic lenses, gonioprism lenses, condensing lenses, ophthalmic prism lenses, prism bars, contact lens, and the like. The eye care accessories 5 considered herein may also include one or more accessories that indirectly contact a patient's eye surface in that they make direct contact with a tool, fluid, or device that directly contacts the eye surface, such as ophthalmic medication containers such as eyedropper bottles, eyedropper bottle caps, nozzles of an eyedropper bottle, contact lens cases, and the like. The eye care accessories 5 considered herein may also include one or more accessories that do not contact the patient's eye surface directly or indirectly, but are used near the eyes, including eyeglasses, eyeglass cases, near vision screening cards, handheld occluders, color vision plates, and the like.

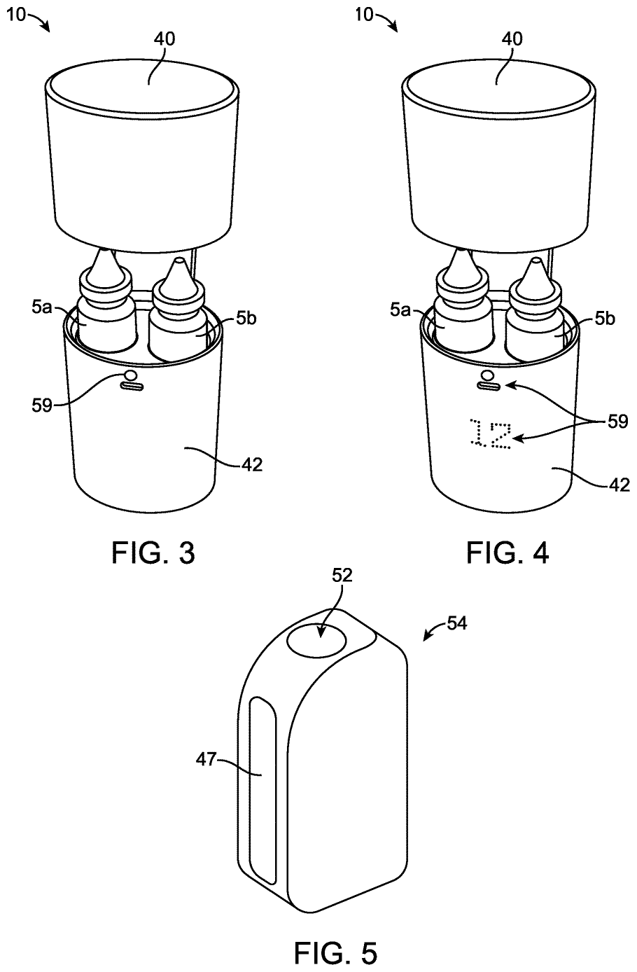

As mentioned above, each of the devices described herein can incorporate at least one treatment bay 52 that is sized and shaped to receive an eye care accessory 5 that is to be disinfected by the device 10. The device 10 can include one, two, three, four, or more treatment bays 52. FIGS. 2E-2F shows a device 10 having a single treatment bay 52 for an eye care accessory 5, which is an eye dropper bottle. FIGS. 3 and 4 show devices 10 having two treatment bays 52 each sized and shaped to receive an eye care accessory 5 that is an eye dropper bottle. FIGS. 6A-6D show a device 10 having three treatment bays 52 each sized and shaped to receive an eye care accessory 5. The eye care accessories 5 can have the same or different form factors from one another. For example, FIGS. 2E-2F illustrate two different sized eye dropper bottles received within the treatment bay 52 of the device 10. As another example, FIG. 6B shows an eye dropper bottle in a first treatment bay 52a, a goniolens in a second treatment bay 52b, and a tonometry device in a third treatment bay 52c.

In some implementations, such as shown in FIG. 2F or FIGS. 3-4, the treatment bay 52 is formed as a hollow receptacle having a lower surface 44 that is sized larger than a maximum outer diameter of a lower end of the eye care accessory 5 being supported and at least one side wall 47 that is sized to surround a portion of a circumference of the accessory 5. The at least one side wall 47 extends along at least a lower end region of the accessory 5 (e.g., ½ up to about ⅔ of the total length of the accessory) to aid in maintaining the accessory 5 in a desired orientation within the treatment bay 52. The treatment bay 52 can, but need not, fully enclose the eye care accessory 5. For example, one of the treatment bays 52 in the device 10 shown in FIGS. 2D-2F has a side wall 47 that lies adjacent to only one side of the accessory 5, however the treatment bay 52 can form a fully cylindrical receptacle that surrounds the full circumference of the lower end of the accessory 5. The shape of the side wall(s) 47 can vary depending on the shape of the eye care accessory 5 being supported. The side wall 47 of the treatment bay 52 shown in FIG. 2D is substantially circular and forms a cylindrical receptacle or slot, which is shaped to receive a correspondingly cylindrical bottle. The geometric shape of the treatment bay 52 formed by the side walls 47 can but need not correspond specifically to the geometric shape of the accessory 5 and can have any of a variety of geometric cross-sectional shapes (e.g., circle, oval, semi-circle, triangle, square, rectangle, pentagon, hexagon, octagon, etc.).

Again with respect to FIGS. 6A-6D, which illustrate an implementation of the device 10 having three treatment bays 52 that each enclose an eye care accessory 5. Each treatment bay 52 includes a lower surface 44 that can support a corresponding lower surface of the eye care accessory 5 received within the treatment bay 52. For example, an eye dropper bottle can be supported on its lower surface to ensure the bottle remains in an upright (or near upright) position within the treatment bay 52 during use. The treatment bays 52 can include at least a first side wall 47 and at least a second side wall 47 where each of the side walls 47 includes at least one light source 22. Preferably, each side wall 47 incorporates more than one light source 22, which will be described in more detail below. The side walls 47 can separate one treatment bay 52 from another. The distance between the side walls 47 across a treatment bay 52 defines a maximum outer diameter of the eye care accessory 5 that can be received within the treatment bay 52. The lower surface 44 of the treatment bay 52 can be adjusted as described elsewhere herein, such as by adding an adaptor or with an adjustment mechanism to change a relative location of the lower surface 44 within the base 42 to ensure the target surface of the accessory to be treated is optimally positioned relative to the light sources 22 of the treatment bay 52 (i.e., so the target contact surface of the accessory 5 is within the threshold distance range of a light source 22). The treatment bay 52 can also incorporate an upper surface or ceiling 45 located over the lower surface 44. The ceiling 45 can include at least one light source 22, which may also be described herein as a ceiling light or chandelier LED. The treatment bay 52 provides full coverage of the eye care accessory 5 between the lower surface 44 and the ceiling 45 and between the side wall(s) 47.

For certain eye care accessories 5, such as eyedropper bottles, the positioning provided by the device 10 maintains the accessory 5 in an upright vertical position to prevent spillage during use. The arrangement of the treatment bay 52 within the device 100 can be designed to ensure the accessory 5 is within a fully upright vertical position, or within 100 degrees of the vertical position, such as within 60 degrees of the vertical position, or within 15-45 degrees of the vertical position. The eye care accessories 5 can include an eyedropper bottle, which is in the capped or uncapped state, while the bottle remains near vertical or within about 60 degrees of the vertical axis. FIGS. 2D-2F illustrate the device 10 with the lid 40 moved away from the base 42 exposing an interior cavity 50 defined by the housing 12. The accessory 5 is shown in FIGS. 2E-2F held upright within the housing 12. A first treatment bay 52a of the base 42 is sized to receive a first accessory 5a (e.g., an eye dropper bottle) and a second treatment bay 52b of the base 42 is sized to receive a second accessory 5b (e.g., the cap of the eye dropper bottle). The size and shape of the treatment bays 52 in the base 42 as well as the interior cavity 50 of the device 10 overall can vary to accommodate and support any of a variety of eye care accessories 5.

The device 10 can accommodate a complete size range of manufactured eye care accessories, for example, eye dropper bottles manufactured from the smallest size (e.g., 2.5 mL or smaller) up to larger sizes (e.g., about 15 mL). In some implementations, one or more adaptors 54 (see FIG. 5) can be positioned inside the housing 12 that aid in holding, storing, and disinfecting other types of devices within the same housing 12. For example, a first adaptor 54 can be positioned within the housing 12 that incorporates one or more treatment bays 52 sized and shaped to mate with and/or support a first accessory 5, such as an eye dropper bottle cap. A first adaptor 54 can be removed from the housing 12 and replaced by a second adaptor 54 that incorporates one or more treatment bays 52 sized and shaped to mate with and/or support a second accessory 5, such as a second eye dropper bottle. Thus, when the first adaptor 54 is in position within the housing 12, the device 10 can support an eye dropper bottle and its cap whereas when the second adaptor 54 is positioned within the housing 12, the device 10 can support two eye dropper bottles. The inner surfaces of the adaptor 54 (the surfaces defining the one or more treatment bays 52) can be sized and shaped to mate with the accessory 5 being supported. The outer surfaces of the adaptor 54 can be sized and shaped to mate with inner surfaces of the housing 12 so that the adaptors 54 remain in place within the housing 12 during use, but can be removed by a user when an alternative arrangement of adaptors is desired. The combination of adaptors 54 within the housing 12 can vary so that any combination of accessories 5 can be held within the device 10, as described elsewhere herein.

The device 10 need not rely upon adaptors 54 to provide a proper fit with an accessory 5 and/or to ensure the position and radiant intensity of the light sources 22 are providing the desired direct light exposure of each accessory 5 within the interior. In still further implementations, the device 10 can additionally or alternatively incorporate an adjustor 48 configured to move a portion of the treatment bay 52, such as the lower surface 44 relative to the base 42, for example, to bring the nozzle of an eye dropper bottle to within a specified distance from the UV-C light source 22. This provides consistent UV-C light exposure for a variety of sized bottles within the same housing 12. The configuration of the adjustor 48 can vary including any of a variety of linear displacement mechanisms. FIGS. 2A-2F illustrates an adjustor 48 that is in the form of a knob located under the base 42 of the housing 12. Rotation of the knob in a first direction can raise the lower surface 44 and rotation of the knob in a second, opposite direction can lower the lower surface 44 to change the distance between an eye accessory supported by the lower surface 44 and the UV light source 22 within the housing 12. It should be appreciated that the lower surface 44 need not be adjustable to deliver consistent radiation to the accessory 5 within the treatment bay 52. Instead, full disinfection can be achieved through the positioning of angled surfaces and/or incorporation of additional light sources 22 without incorporating an adjustor 48 for the lower surface 44, which is described in more detail below.

Each of the plurality of lights sources 22 is arranged relative to the treatment bays 52 so that the contact surfaces of the accessory 5, including one or more of side surface, upper surface, and lower surface, are directly exposed to UV light with minimal to no shadowing. A minimum distance between the light sources 22 and the accessory 5 within the treatment bay 52 is selected to allow the accessory 5 to be received within the treatment bay 52 and allow a user's fingers to access the accessory 5 for insertion/removal of the accessory 5 to/from the treatment bay 52. As an example, the treatment bay 52 may be sized to receive the largest conventional ophthalmic accessory (e.g., 40 mm Katena ophthalmic lens as shown in FIG. 6D) while still maintaining a sufficient distance between the accessory 5 and the light source 22 within the side walls 47 of the treatment bay 52. The distance between the light source(s) 22 and the target contact surface of the eye accessory 5 being treated, referred to herein as a threshold distance or threshold distance range, can vary. The threshold distance range can be about 1 mm to about 1000 mm, and is preferably less than about 12 inches or about 1 mm-300 mm, or about 5 mm-60 mm, or about 15 mm-60 mm, or about 15 mm-40 mm, or about 10 mm-40 mm, and any of a variety of distances in between the recited ranges, including about 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 45 mm, 50 mm, 55 mm, 60 mm, 65 mm, 70 mm, 75 mm, 80 mm, 85 mm, 90 mm, 95 mm, or 100 mm. In some implementations, the distance between the target contact surface being treated and the light source 22 is preferably about 40 mm.

As mentioned, eye care accessories 5 typically have surfaces that are intended to make contact with a patient's eye (referred to herein as “target surface” or “contact surface” or “essential surface” and the like) and other surfaces that are not intended to come into contact with a patient's eye, such as a handle or other portion of the accessory that a doctor handles during use of the accessory 5. Each eye care accessory 5 is positioned within the interior of the treatment device 10 so at least the contact surfaces of the accessory 5 are directly exposed to the UV light from the light source(s) 22 without shadowing and/or loss of efficacy. Other surfaces of the accessory 5 besides the contact surfaces (e.g., handle, housing, etc.) may also be treated as the target surface of the direct exposure to UV light. The radiant flux, number, position, and/or orientation of the UV light sources 22 within each treatment bay 52 is designed to provide 360-degree coverage to achieve sanitization, preferably disinfection, more preferably high-level disinfection of the eye care accessory 5 located within the treatment bay 52. The plurality of UV light sources 22 are oriented relative to the treatment bay 52 forming banks 51 of light sources 22 that preferably create multiple overlapping cones of light to eliminate shadowing and ensure adequate exposure of the accessory 5, particularly the contact surfaces of the accessory 5, positioned within the treatment bay 52.

In some implementations, the treatment bay 52 incorporates at least three banks 51 of light sources 22 positioned 120 degrees apart around each treatment bay 52. Preferably, each treatment bay 52 incorporates at least four banks 51 of light sources 22 that are spaced 90 degrees apart around each treatment bay 52 to ensure direct light exposure of the accessory 5 within the treatment bay 52. Each bank 51 can include at least two light sources 22 separated from one another in a vertical direction. A first light source 22 of the at least two light sources 22 can be positioned near an upper end region of the treatment bay 52 and a second light source 22 of the at least two light sources 22 can be positioned near a lower end region of the treatment bay 52. The positioning of the pair of light sources 22 within each bank 51 mitigates shadowing of the upper and lower surface features on the target object positioned within the bay 52. As an example, a dropper bottle may incorporate threads near the nozzle (e.g., within an upper ⅓ of the bottle height) that have upper and lower surfaces. The lower surfaces of the threads may experience shadowing if the light source 22 was near an upper end region of the treatment bay 52 or upper surfaces of the threads may experience shadowing if the light source 22 was near a lower end region of the treatment bay 52 (see FIG. 6C). Similarly, light sources 22 placed only along the side walls 47 of the treatment bay 52 and not at a ceiling 45 of the treatment bay 52 to provide strong exposure to the top face of the accessory 5, would not reliably disinfect all surfaces of the bottle threads. The pair of light sources 22 within each bank 51 of lights mitigate shadowing to ensure the upper and lower surfaces of the object, whether bottle threads, or applanation tips, or tonometer tips, etc. are exposed to light with sufficient intensity for disinfection.

Lambert's Cosine Law says that the radiant intensity from an ideal diffusing reflecting surface is directly proportional to the cosine of the angle between the direction of incident light and normal to the surface. As this angle of incidence from a cone of light increases, the maximum intensity decreases. As such, the device can additionally incorporate at least one light source 22 in the ceiling 45 (e.g., a ceiling or chandelier LED) of the treatment bay 52 to emit light from above an upper surface of the eye care accessory 5 being treated in addition to the UV light sources 22 positioned within each side wall 47 to emit light towards a side surface of the eye care accessory being treated. The ceiling light source 22 can have a greater radiant flux compared to the radiant flux of the light source 22 positioned within the side walls 47 of the treatment bay 52.

In an implementation, each treatment bay 52 has four side walls 47 and a ceiling 45. Each of the ceiling 45 and four side walls 47 can include at least 1 light source 22 and, in the case of the side walls 47, preferably at least 2 light sources 22. Thus, each treatment bay 52 includes at least five light sources 22, preferably more, including six, seven, eight, nine, ten, eleven, or twelve light sources 22 per treatment bay 22. In other implementations, each treatment bay 52 can includes at least one light source 22, preferably a minimum of three light sources 22 per treatment bay 52, and more preferably nine light sources 22 per treatment bay 52. In an implementation, the device 10 includes three treatment bays 52, having four banks 51 per treatment bay 52, and at least two and preferably three light sources 22 per bank 51.

FIG. 6B shows a first bank 51a of light sources 22 within a first side wall 47a and a second bank 51b of light sources 22 within a second side wall 47b. Each of the banks 51 can include preferably at least two light sources 22, including two, three, four, five, six, seven, etc. LEDs within each side wall 47. The implementation of FIG. 6B shows four light sources 22 within each side wall 47 of a treatment bay 52. The implementation of FIG. 6C shows three light sources 22 within each side wall 47 of a treatment bay 52.

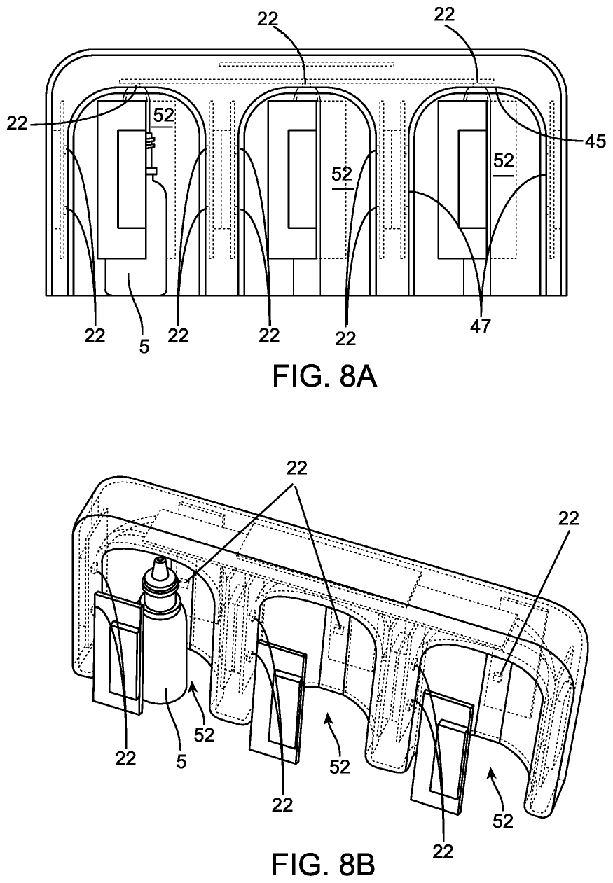

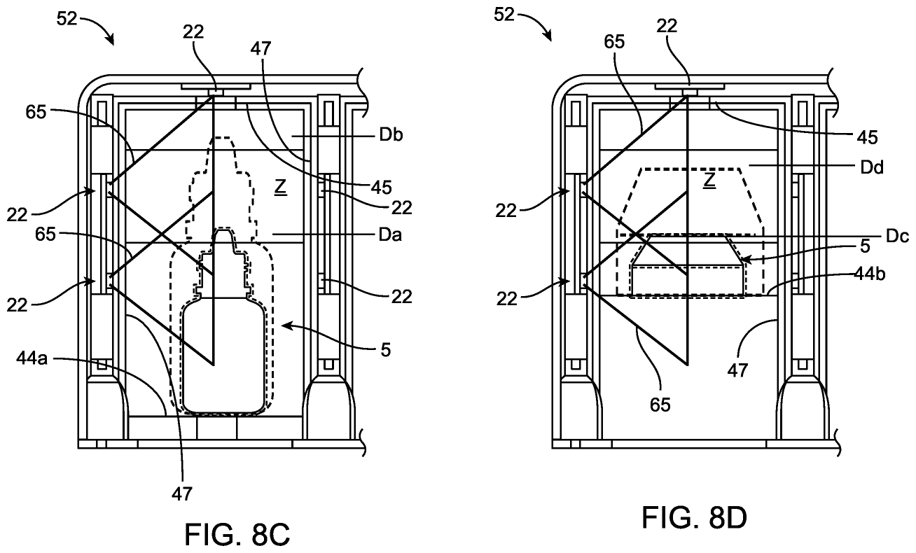

Each light source 22 of the devices described herein is an LED that emits a strong central cone of light that is at least about 60 degrees, preferably about 80 degrees. FIGS. 8A-8B illustrate cones of light 65 emitted by a plurality of light sources 22 within a treatment bay 52. UV-C LEDs are directional light sources, and each produces a focused cone of light 65 that concentrates light within a specific angle. The angle of the cone of light 65 can be about 120 degrees. The maximum light intensity is within the central 80 degrees of the cone 65 and is most intense at about 30 degrees. As the angle increases, the relative intensity drops.

FIGS. 8A-8B show partial views of a disinfection device having three treatment bays 52, each bay 52 including four side walls 47 and a ceiling 45. Each of the side walls 47 includes at least a pair of LEDs 22 arranged in a bank (e.g., located on a printed circuit board) so that the light sources are separated vertically from one another. Each ceiling 45 has a LED light source 22. Each bay 52 can vary in geometric cross-sectional shape (e.g., circle, oval, semi-circle, triangle, square, rectangle, pentagon, hexagon, octagon, or irregular, etc.) and can include at least one side wall forming the geometric cross-sectional shape of the bay 52.

The treatment bay 52 is designed to ensure the target surface(s) of an accessory 5 is positioned within at least one focused cone of light 65 of the LED and within a threshold distance from the light sources 22. FIG. 8C shows one treatment bay 52 having four side walls 47 (front side wall not visible in the figure), a ceiling 45, and a lower surface 44a positioned to support the accessory 5. The lower surface 44a can be arranged to support different sized accessories 5. For example, FIG. 8C shows an accessory 5 that is an eye dropper bottle having a height that, when the bottle is positioned on the lower surface 44a, the top of the nozzle extends to a first distance Da away from the ceiling LED 22. The accessory can be a larger eye dropper bottle so that when the bottle is positioned on the lower surface 44a, the top of the nozzle of the eye dropper bottle extends to a second distance Db away from the ceiling LED 22, the second distance Db being closer to the ceiling LED 22. The distance between the lower surface 44 and the ceiling 45 of the treatment bay 52 is designed so that the top of the nozzle in either size eye dropper bottle is positioned within the threshold treatment zone Z for the ceiling LEDs. Additionally, a distance between the lower surface 44 and the light sources 22 in the side wall 47 is designed so that essential surfaces, such as the threads of the eye dropper bottle, are located within a focused cone of light 65 provided by an LED in a side wall 47. Each distance Da, Db is within the preferred threshold treatment zone Z for the ceiling LEDs and the target surface(s) of the accessory 5, such as the threads and nozzle, is within at least one focused cone of light 65 of an LED 22 in the side wall 47, such as an 80-degree cone of an LED in the side wall 47. The threshold distance can vary depending on the power of the LED. For example, the ceiling LED 22 may be a 52 mW LED having a threshold distance of at least 15 mm and no more than about 40 mm away from the target surface.

FIG. 8D shows an accessory 5 that is an ophthalmic lens having a height that, when the lens is positioned on the lower surface 44b, the top of the lens extends to a first distance Dc away from the ceiling LED 22. The lower surface 44b can be formed as an adaptor, shelf, or other modular feature that lifts the location of the lower surface 44 relative to the ceiling LED 22. Ophthalmic lenses come in different sizes and shapes akin to eye dropper bottles. Thus, a larger lens positioned on the lower surface 44b positions the top of the lens a second distance Dd away from the ceiling LED 22, the second distance Dd being closer to the ceiling LED 22 than the first distance Dc for the smaller lens. The distance between the lower surface 44 and the ceiling 45 of the treatment bay 52 is designed so that the top of the lens regardless of overall lens size is positioned within the threshold treatment zone Z for the ceiling LEDs. Additionally, a distance between the lower surface 44 and location of the light sources 22 in the side wall 47 is designed so that essential surfaces of the lens, such as the surface intended to contact the anterior surface of the eye, are located within a focused cone of light 65 provided by an LED in a side wall 47. Each distance Dc, Dd is within the preferred threshold treatment zone Z for the ceiling LEDs and the target surface(s) of the accessory 5, such as the surface of the lens that contacts an anterior surface of a patient's eyeball, is within at least one focused cone of light 65 of an LED 22 in the side wall 47, as discussed elsewhere herein.

The LEDs 22 in the treatment bay 52 create cones of light 65 so that multiple overlapping cones of light 65 within each treatment bay 52 are created. FIGS. 8C-8D show only two overlapping cones of light 65 as an illustration and to provide clarity although it should be appreciated that more than these two cones of light 65 may be present depending on the total number of LEDs incorporated within a single treatment bay 52. As discussed elsewhere herein, the light sources 22 located within the side walls 47 can include an upper LED separated vertically from a lower LED. The upper LED creates an upper cone of light 65 and the lower LED creates a lower cone of light 65. The upper and lower cones of light 65 create an overlap. The distance between the lower surface 44 and the ceiling 45 as well as the distance between the lower surface 44 and the light sources in the side walls 47 ensures the target surfaces of the eye care accessory are within the threshold treatment zone of the ceiling LED and within the focused cone of light 65 of at least one side wall LED 47. The lower cone of light 65 is useful in illuminating a first region of the accessory, such as a lower surface of a thread or surfaces of a shorter accessory, and the upper cone of light 65 alone or in combination with the ceiling LED is useful in illuminating another region of the accessory, such as an upper surface of a thread or surfaces of a taller accessory. The distance of the target surface(s) is preferably maintained within the threshold treatment zone for the LEDs to be directly exposed to the preferred threshold energy delivery of that LED (e.g., 160 mJ/cm²).

The treatment bay 52 can have a different number of side walls 47 than described above (e.g., eight side walls 47 arranged in an octagon shape). The shape of the bay 52 and arrangement of light sources 22 within each bay 52 is designed to maximize first-order exposure of the eye care accessory 5 to the UV light (i.e., direct exposure within the threshold distance) within the treatment bay 52 from each UV-C LED light source 22 and ensures coverage over the entire accessory 5 even if the accessory 5 is slightly mispositioned within the treatment bay 52.

The distance between the side walls 47 and the distance between the ceiling 45 and lower surface 44 ensures the target surfaces of the accessory 5 are within the preferred zone Z or threshold distance away from the light sources 22 including at least some finger space for a user to position the accessory 5 within the bay 52 and remove the accessory 5 from the bay 52. The power of the LEDs 22 can vary depending on the threshold distance the target surface(s) of the accessory 5. For example, the LEDs in the banks of light can have a radiant flux between 0.1 mW to 300 mW. However, the LEDs in the banks of light can be lower powered, such as about 10-15 mW, if the distance between the light source and the target surface is shorter (e.g., about 10 mm). Lower power LEDs are preferred because the cost of manufacturing is reduced. The power of the LED, however, cannot be so low that the threshold distance is impractical. An LED that is just 5.75 mW in radiant flux may need the accessory to be as close as about 10 mm, but the angle of the cone of light 65 at that distance may be large enough to disinfect only very small accessories and would be less suitable for larger accessories 5.

Table 1 below illustrates energy emitted (mW/cm²) by a light source that is a 5.75 mW UV-C LED over 180 seconds and the energy received (mJ/cm²) by a target surface area for disinfection. The maximum distance from the light source to the target surface area estimated to achieve the threshold energy delivery of 160 mJ/cm² was about 14 mm.

TABLE 1

| UV-C | Distance | Energy | Energy |
|---|---|---|---|
| radiation | from | received by | received by |
| Intensity | LED | 4 mm area | 20 mm area |
| (mW/cm²) | (mm) | (mJ/cm²) | (mJ/cm²) |
| 3.591 | 7 | 646.42 | |
| 1.760 | 10 | 316.75 | |
| 0.782 | 15 | 140.78 | 140.78 |
| 0.440 | 20 | 79.19 | 79.19 |
| 0.282 | 25 | 50.68 | 50.68 |
| 0.196 | 30 | 35.19 | 35.19 |
| 0.144 | 35 | 25.86 | 25.86 |
| 0.110 | 40 | 19.80 | 19.80 |
| 0.087 | 45 | 15.64 | 15.64 |
| 0.070 | 50 | 12.67 | 12.67 |

Table 2 below illustrates energy emitted (mW/cm²) by a light source that is a 10 mW UV-C LED over 180 seconds and the energy received (mJ/cm²) by a target surface area for disinfection. The maximum distance from the light source to the target surface estimated to achieve the threshold energy of 160 mJ/cm² was about 18 mm.

TABLE 2

| UV-C | Distance | Energy | Energy |
|---|---|---|---|
| radiation | from | received by | received by |
| Intensity | LED | 4 mm area | 20 mm area |
| (mW/cm²) | (mm) | (mJ/cm²) | (mJ/cm²) |
| 6.246 | 7 | 1124.22 | |
| 3.060 | 10 | 550.87 | |
| 1.360 | 15 | 244.83 | 244.83 |
| 0.765 | 20 | 137.72 | 137.72 |
| 0.490 | 25 | 88.14 | 88.14 |
| 0.340 | 30 | 61.21 | 61.21 |
| 0.250 | 35 | 44.97 | 44.97 |
| 0.191 | 40 | 34.43 | 34.43 |
| 0.151 | 45 | 27.20 | 27.20 |
| 0.122 | 50 | 22.03 | 22.03 |

Table 3 below illustrates energy emitted (mW/cm²) by a light source that is a 40 mW UV-C LED over 180 seconds and the energy received (mJ/cm²) by a target surface area for disinfection. The maximum distance from the light source to the target surface estimated to achieve the threshold energy of 160 mJ/cm² was about 37 mm.

TABLE 3

| UV-C | Distance | Energy | Energy |
|---|---|---|---|
| radiation | from | received by | received by |
| Intensity | LED | 4 mm area | 20 mm area |
| (mW/cm²) | (mm) | (mJ/cm²) | (mJ/cm²) |
| 24.983 | 7 | 4496.86 | |
| 12.241 | 10 | 2203.46 | |
| 5.441 | 15 | 979.32 | 979.32 |
| 3.060 | 20 | 550.87 | 550.87 |
| 1.959 | 25 | 352.55 | 352.55 |
| 1.360 | 30 | 244.83 | 244.83 |
| 0.999 | 35 | 179.87 | 179.87 |
| 0.765 | 40 | 137.72 | 137.72 |
| 0.605 | 45 | 108.81 | 108.81 |
| 0.490 | 50 | 88.14 | 88.14 |

Table 4 below illustrates energy emitted (mW/cm²) by a light source that is a 52 mW UV-C LED over 180 seconds and the energy received (mJ/cm²) by a target surface area for disinfection. The maximum distance from the light source to the target surface estimated to achieve the threshold energy of 160 mJ/cm² was about 42 mm.

TABLE 4

| UV-C | Distance | Energy | Energy |
|---|---|---|---|
| radiation | from | received by | received by |
| Intensity | LED | 4 mm area | 20 mm area |
| (mW/cm²) | (mm) | (mJ/cm²) | (mJ/cm²) |
| 32.477 | 7 | 5845.92 | |
| 15.914 | 10 | 2864.50 | |
| 7.073 | 15 | 1273.11 | 1273.11 |
| 3.978 | 20 | 716.13 | 716.13 |
| 2.546 | 25 | 458.32 | 458.32 |
| 1.768 | 30 | 318.28 | 318.28 |
| 1.299 | 35 | 233.84 | 233.84 |
| 0.995 | 40 | 179.03 | 179.03 |
| 0.786 | 45 | 141.46 | 141.46 |
| 0.637 | 50 | 114.58 | 114.58 |

In some implementations, the LEDs 22 in the side walls 47 are lower powered than the LEDs 22 in the ceiling 45. For example, the side wall 47 LEDs 22 can have a radiant flux that is between about 10-15 mW and the ceiling 45 LEDs 22 can have a radiant flux that is greater than about 15 mW, such as between about 20-60 mW. The higher-powered ceiling LED increases the distance away the accessory 5 may be positioned and thus, increases the flexibility of the treatment bay 52 for a variety of accessory types and sizes.

The treatment bays 52 of the devices described herein can allow for a threshold distance (i.e., distance between a light source 22 in the treatment bay 52 and the target surface of the accessory to be irradiated) that is about 1 mm-1000 mm, or 10 mm-100 mm, preferably about 20 mm-50 mm. The light source 22 and the target surface of the eye care accessory 5 are separated by a minimum distance to ensure direct exposure of the target surface by maximum radiant flux emitted from the light source 22. A maximum distance between the light source 22 and the target surface is based on the radiant flux of the UV-C LED. The maximum distance is affected by the power of the LED, disinfection cycle length, and threshold dose needed for disinfection. The furthest distance a target surface may be positioned from even a high-powered LED source may be considerably small (e.g., less than 100 mm) to achieve a threshold energy delivery that is at least 160 mJ/cm² within a 3-minute treatment cycle (i.e., target for high-level disinfection).

As discussed elsewhere herein, the high-level disinfection of each eye care accessory 5 can be achieved without relying on any reflected light or reflective surfaces within the interior cavity of the device 10. Using reflective materials like aluminum or Teflon may improve the total irradiance inside a disinfection chamber, but the additional distance traveled by the UVC light significantly weakens the efficacy of reflected light according to the inverse square law, which says the energy twice as far from the source is spread over four times the area, hence one-fourth the intensity. Reflectors functionally double or triple the distance from the light source. For example, even when an object is positioned within a relatively short distance from a light source (e.g., 20 mm), the total distance traveled by light after reflection can be as about 68 mm within the chamber. This increased travel distance for reflected UV-C light significantly drops in intensity of radiation to areas of the target object only indirectly exposed to light resulting in hot and cold spots and poor uniformity of coverage.

Each light source 22 in the treatment bays 52 can be a UV-C LED having an 80-degree cone of maximum radiant flux. The minimum distance between the light source 22 and the target surface of the accessory 5 to be irradiated for disinfection (e.g., an ophthalmic lens that is about 20 mm across the lens face) can be generally no less than about 15 mm based on the spread of emitted light and the size of the contact surface of the accessory to achieve direct exposure. This distance accounts for some user error (~5 mm) when centering the lens in the treatment bay 52. A contact surface having a diameter that is greater than about 20-25 mm (e.g., 40 mm Katena lens) may be positioned greater than 15 mm from the light source 22 to receive sufficient direct light exposure whereas smaller contact surfaces (e.g., applanation tips, iCare tonometer tips, and eyedropper bottle nozzles) may be positioned closer than 15 mm from the light source 22. Some smaller objects may be positioned as close as about 7 mm away from the light source 22 in the treatment bay 52.

Ophthalmic eye care accessories 5 accommodated by the devices described herein can vary widely in shape and size and include one or more of eyedropper bottles, eyedropper bottle caps, eyeglasses, eyeglass cases, contact lens, contact lens cases, applanation tonometer prism tips, rebound tonometer probes, ophthalmic lenses, gonioprisms, condensing lenses, near vision screening cards, handheld occluders, color vision plates, ophthalmic prisms, prism bars, and the like. As an example, ophthalmic gonioscopy and laser lenses that can be disinfected using the devices described have a contact diameter (i.e., the diameter of the surface that is intended to be placed against a patient's eye and that is the target surface to be irradiated for disinfection) that can be in a range of about 8 mm to about 20 mm. The lenses also have an overall width/diameter and height that is in a range of about 20 mm-40 mm and a height is in a range of about 15 mm-35 mm. Some lenses have a grip/rim height that is as small as 5 mm-10 mm, whereas others have attached handles that create a total height of about 100 mm-120 mm. The treatment bays 52 of the devices described herein can be designed to receive accessories at least within these size ranges. Dimensions outside these example range are considered as well.

As discussed elsewhere herein, the eye care accessory 5 can include eye dropper bottles and/or caps. Table 5 lists a variety of eye dropper bottles that can be disinfected using the devices described herein. All dimensions are provided in mm unless otherwise noted. Eye dropper bottles can be manufactured in volumes of about 2 mL-5 mL or larger than 5 mL, such as 10 mL-15 mL. The eye dropper bottles regardless the total volume it contains can have an overall diameter in a size range of about 20 mm-30 mm. The eye dropper bottles have an overall height when the cap is engaged with the bottle that can be in a size range of about 45 mm-80 mm. The height can be less (e.g., by about 0.5 mm-4 mm) when the cap is not in place over the nozzle. The devices described herein can incorporate treatment bays 52 that are sized and shaped to receive eye dropper bottles at least within these size ranges. Dimensions outside these example range are considered as well.

TABLE 5

| | | Height | Height |
|---|---|---|---|
| Accessory | Diameter | (w/cap) | (w/o cap) |
| Dorzolamide 10 mL | 25 | 59 | 56.6 |
| (Micro Labs USA Inc) | | | |
| Proparacaine 15 mL | 25.4 | 78.2 | 77.4 |
| (Bausch & Lomb) | | | |
| Tropicamide 15 mL (Somerset) | 25.4 | 77.6 | 77.25 |
| Phenylephrine 15 mL | 24.4 | 77.6 | 76.7 |
| (Lifestar Pharma LLC) | | | |
| Ketorolac 10 mL (Apotex Inc) | 22.6 | 65.84 | 64.8 |
| Prednisolone acetate | 25.2 | 56.2 | 54.5 |
| 5 mL (Allergan Inc) | | | |
| Simbrinza 2.5 mL (Alcon Inc) | 20 | 48.85 | 48 |
| Moxifloxacin 3 mL (Sandoz) | 24.7 | 53.1 | 52.2 |

The specific wavelengths, intensities, radiant flux, duty cycles, frequencies of the UV light sources 22 can vary. The UV light source 22 can be tailored to provide energy in very narrow bands including, for example, UV-C. Output spectrums can be tailored to provide total spectrums within, for example, ±500 nm, ±100 nm, ±10 nm or ±1 nm or less of the center band frequency of about 250-290 nm or 255-280 nm. The dose may be a timed exposure at a controlled power to destroy biocontaminants including viruses, bacteria, spores, fungi, molds, protozoa, and yeasts, for example. For example, the UV light source 22 is preferably a UV-C LED. UV-C LEDs can be cycled at short intervals, have a relatively small size, and are generally low powered rendering them suitable for portable devices, such as the desktop and pocket-sized devices described herein. The UV-C LEDs are capable of being powered on and off multiple times each day to perform a multitude of treatment cycles without negatively impacting the light source. For example, the devices described herein are designed to perform at least one treatment cycle a day (i.e., light sources powered on and off during a single treatment cycle) up to at least 100 cycles each day, on average about 20 to about 50 treatment cycles per day, (or just 1-3 times a day for a personal eyedropper bottle disinfection device) without negatively impacting the light source(s) 22 within the device. In some implementations, the UV-C LED can irradiate the eye care accessory 5 for between 1-5 minutes at a frequency of 50-1000 Hz and a duty cycle that varies between 10-100%.

In contrast, UV-C mercury-vapor lamps are high-flux UV light sources with longer heat/cool times in comparison to UV-C LEDs. UV mercury lamps presently available have long heat/cool times that prevent their ability to be pulsed and limit their practical use in disinfection of eye care accessories for ophthalmology use. The cycling should be relatively short for ophthalmology office use (e.g., less than 5-minute cycling or exposure period to disinfect) and can be repeated multiple cycles a day (e.g., over 100 cycles per day), which would cause a UV-C mercury-vapor lamp of present technology to fail. Known UV-C mercury-vapor lamps are large-sized bulbs that are impractical for portable devices, such as a device that is designed to be held within a user's hand, pocket, or purse, or a device designed to have a small footprint within an optometry or ophthalmology office. The UV light source considered herein is one that need not have long cool-down/heat-up times and are capable of being pulsed. Any UV light source, such as UV-C LEDs, which are capable of being used in this manner, are considered herein as the light source to disinfect the eye care accessories described herein.

The light sources 22 of the devices described herein can be a UV-C LED that is within a range of wavelengths to achieve a dose of irradiating energy within a treatment cycle that achieves high-level disinfection of each accessory. High-level disinfection achieved means at least 6 log reduction of bacteria and at least 4 log rejection of viruses. For ophthalmic devices, the threshold UV-C dose to achieve high-level disinfection is 160 mJ/cm², which is sufficient to achieve 4 log reduction in pathogenic ocular viruses including ophthalmic adenovirus serotypes, which are among the most resistant organisms associated with ophthalmic infections. Achieving 6 log reduction of pathogenic ocular bacteria may require less (e.g., maximal energy of 60 mJ/cm²). The range of wavelength of the UV-C LED is preferably between 200 nm-280 nm (e.g., 275 nm wavelength of UV-C light) and the treatment cycle is preferably less than about 5 minutes, such as about 3 minutes or as short as about 1 minute. Each light source 22 within the interior of the device is positioned within a threshold distance range from the target surface of the accessory 5 being treated for the purposes of disinfection of the target surfaces.

UV-C LED light sources are manufactured with radiances that vary, for example, from 1 mW-300 mW. Factors that influence how radiant flux of a UV-C LED light source affects the target surface being treated include the 1) distance between the light source and the target surface; 2) duration of exposure; and 3) angle of incident light. The irradiance declines as distance between the light source and the target surface increases. The decline of irradiance follows an inverse square law and is not evenly distributed as with a point source of light. An inverse-square law is one where the observed “intensity” of a specified physical quantity is inversely proportional to the square of the distance from the source of that physical quantity. In mathematical notation, the inverse square law can be expressed as an intensity (i) varying as a function of distance (d) from a center. Intensity is proportional to the reciprocal of the square of the distance. LEDs use primary optics that direct a cone of light of approximately 120 degrees, with the central 60-80 degrees containing most of the usable energy.

The UV light sources 22 of the devices described herein can have a radiant flux of 0.1-1000 mW, more preferably 1 mW-100 mW. “Radiant flux” as used herein is a measurement to describe the light source itself and refers to the total amount of radiant energy emitted by a light source per unit of time. Radiant flux quantifies the overall power output of the UV-C light source in terms of electromagnetic radiation. Radiant flux is typically measured in watts (W) and can also be described as “brightness” of a light source. “Intensity” as used herein refers to the amount of radiant flux per unit solid angle, a directional quantity. Intensity is expressed in mJ/cm² (light per unit area over a time period).

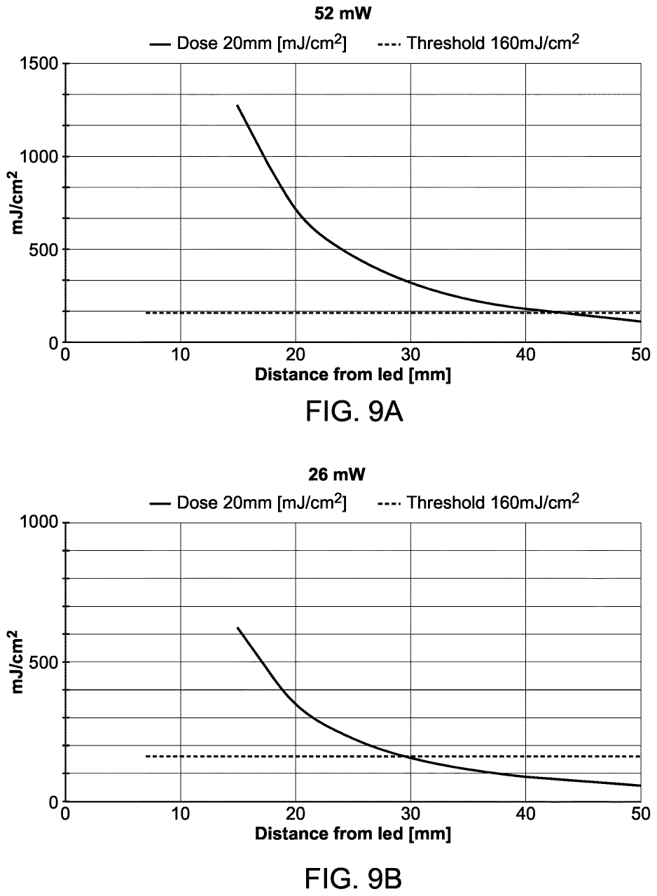

FIG. 9A illustrates the radiant flux of a medium-power LED (53 mW) run for 180 seconds to illustrate the usable range of distances between the light source and an accessory having a diameter of 20 mm (solid line). The minimum distance between the light source and the accessory was about 15 mm. The energy delivered to the target surface of the accessory dropped below the threshold of 160 mJ/cm² (dashed line) at about 42 mm distance from the light source.

FIG. 9B illustrates the radiant flux of a weaker-power LED (26 mW) to illustrate the smaller range of distances to reach the threshold disinfection of 160 mJ/cm² within 180 seconds. The minimum distance between the light source and the accessory having a diameter of 20 mm (solid line) was once again about 15 mm, but the energy delivered to the target surface of the accessory dropped below the threshold of 160 mJ/cm² (dashed line) at about 30 mm distance from the light source.

Preferably, the disinfection of each eye care accessory 5 can be achieved without any reflected light or relying on any reflective surfaces within the interior of the device and instead relies only upon exposure of contact surfaces to direct light from the UV light source(s) 22. However, the irradiating ultraviolet light can be reflected within the housing 12 in some implementations. The interior cavity 50 can be lined circumferentially with a reflective angled surface that causes reflection and re-reflection of communicated UV light. Thus, directly radiated and optionally reflectively radiated light rays from the UV source 22 impact the eye care accessory 5 surface, for example the nozzle, threads, and cap of an eye dropper bottle, to eliminate biocontaminants. One or more surfaces of the interior cavity 50 can be lined with one or more reflective angled surfaces to provide diffuse, wrap-around illumination of the eye care accessory 5 being disinfected without shadowing or loss of exposure. In an example, the treatment bays 52 can be lined with metallized/aluminized reflective surfaces. However, the devices described herein can provide disinfection without shadowing and without any reflective surfaces on an interior of the treatment bay 52.

The ultraviolet radiation can be delivered in pulsed wave form or as continuous radiation. The power can be controlled by modulation of current and/or voltage applied to the UV light source 22 to substantially achieve the controlled radiation level. The pulse frequency and pulse duration can be varied to create desired power. If pulsed, pulses may be delivered at a fixed or variable frequency of between about 50 Hz and about 1 kHz, such as between about 100 Hz and 500 Hz, or between about 200 Hz and about 250 Hz. Individual pulse duration may be less than one second, such as between about 1 ns and 100 ms, or between about 10 ns and 10 ms, or between about 100 ns and 1 ms, or between about 1000 ns and about 0.1 ms, such as between about 300 and 400 microseconds. Pulse frequency, duty cycle, and length will be based on specific application for bactericidal, viricidal, or fungicidal purposes, with a particularly preferred frequency of 50 Hz and duty cycle of 50% applied for approximately 3 minutes.

In some implementations, the eye care accessory 5 is an eye dropper bottle and one or more portions of eye dropper bottle can be made of UV-C transmissible material to allow for pass-through disinfection of the contents of the bottle. For example, the eyedropper cap and/or bottle can be made of UV-C transmissible material so that medications, solutions, and/or parts of the container contained within the bottle whether in the capped or uncapped state are treated with the UV light. The UV-C transmissible material of the cap and/or bottle can be sized and shaped to fit conventional eyedropper bottles. Replacing the manufactured cap formed of materials that are not UV transmissible with a UV-C transmissible cap allows for pass-through disinfection of both the cap and the eyedropper nozzle and threads without needing to leave the bottle uncapped within the device 10 (such as shown in FIGS. 2E-2F). The UV-transmissible eyedropper cap can be provided to the patient or user to replace the original cap on the eyedropper bottle for use with the device 10. This enables direct, pass-through UV illumination of the capped nozzle as well as all surfaces of the cap when secured within the internal cavity of the housing. The UV-C transmissible cap can be disposable, exchangeable, or reusable.

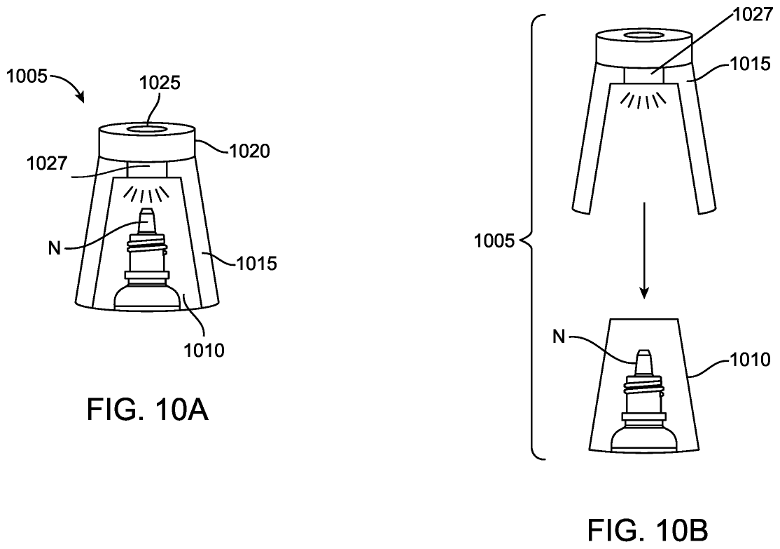

FIGS. 10A-10B illustrates a replacement eyedropper cap 1005 to disinfect a nozzle N of an eyedropper bottle. The cap 1005 can include an inner housing 1010 and an outer shell 1015. The inner housing 1010 is designed to encapsulate the nozzle N and the outer shell 1015 is designed to encapsulate the inner housing 1010. The outer shell 1015 can include one or several UV-C light sources 1020 and/or light pipes to allow for pass-through disinfection of the eyedropper bottle nozzle N in the closed or locked state. The inner housing 1010 can be a UV-C transmissible material allowing for the light from the light source(s) 1020 to pass to the nozzle N. The UV-C transmissible material can include quartz, UV-grade fused silica, calcium fluoride, topaz, artificial diamond, borate crystal, or other material through which UV-C can pass, by at least about 30%. The eyedropper bottle, nozzle, and/or native cap of the eyedropper bottle can also be formed of UV-C transmissible material(s). The outer shell 1015 is preferably impermeable to UV light to prevent leakage of light from the light source 1020 to a user. The cap 1005 can be attached to and removed from the eyedropper bottle such that the cap 1005 mimics the native eye dropper cap and is usable as a cap replacement. The inner housing 1010 can incorporate features to fix the cap 1005 to a region of the eyedropper bottle, including by way of threads, snap lock, interference fit, or other fixation feature, to ensure the cap 1005 stays in position over the nozzle N. The cap 1005 can incorporate a user interface 1025, such as a push button or other type of actuation feature to turn on the light source 1020. The cap 1005 can also include a power source 1027, such as a battery, to power the light source 1020. In some implementations, the outer shell 1015 of the cap 1005 can be removed from the inner housing 1010 of the cap 1005 by a user. For example, the outer shell 1015 can be removed for charging the battery while the inner housing 1010 stays in place over the nozzle. The outer shell 1015, once fully charged, can then be reattached to the inner housing 1010 for further use.

The devices described herein contain the UV light within the interior cavity 50 to protect users from inadvertent exposure to damaging UV rays. For example, the implementation shown in FIGS. 2A-2F has a lid 40 movably connected to the base 42 to reveal at least a portion of the interior cavity. The lid 40 can be moved away from the base 42 to open the device 10 and expose the interior cavity 50, such as to place an accessory 5 within the treatment bay 52, and moved toward the base 42 to close the device and seal the interior cavity 50. Sealing this inner cavity 50 prevents transmission or leakage or escape of UV light and potential harm to the user during use. The lid 40 can be removably attached to the base 42, such as with a hinge 57 (see FIG. 2C and FIG. 6B) or sliding attachment, such as a shutter that slides relative to the base 42 to reveal the treatment bay(s) in the interior cavity 50. The lid 40 need not remain coupled to the base 42 when in the open position. For example, the lid 40 can be fully removable from the base 42, such as with a snap lock, interference fit, threaded, or another engagement (see FIGS. 3-4). Use of a term such as “lid” to refer to the cover of the housing 12 is not intended to be limiting to any particular arrangement of covering. For example, the relative orientation of the lid 40 to the base 42 can vary and need not be arranged on an upper surface of the base 42, but instead can be on side surface, such as a front face of the base 42 to open forward and backward like a door as opposed to up and down like a cap. Generally, the housing 12 of the devices described herein forms an enclosure designed to contain the UV light within the interior of the device during use. The interior cavity 50 including the treatment bay(s) 52 within the housing 12 can be accessed according to any of a variety of mechanisms including a lid, cap, door, shutter, or other type of mechanism that reveals the space when it is desired by a user. The housing 12 is preferably formed of a material that is easy-to-clean, anti-bacterial, and durable. In some implementations, at least a portion of the housing 12 can be magnetic or incorporate a magnetic element that allows attachment of the device 10 to another component to facilitate storage or transport.

Even when the lid 40 is engaged with the base 42 and the device 10 is sealed, a user can view the interior cavity 50 using the at least one window 46. An implementation of the window 46 is shown in FIGS. 2A-2B. The window 46 can be aligned at least in part with a respective treatment bay 52. For example, FIG. 6A illustrates three windows 46 arranged relative to three treatment bays 52. The window 46 can be a clear material (e.g., plastic, glass) that allows for visualization of the eye care accessory 5 contained within the interior cavity, including the color of the accessory within the cavity, without the lid 40 needing to be removed or opened. This allows for the user to identify whether the UV light within the housing 12 is active and currently in use without needing to stop treatment by opening the housing 12. The user is also able to identify what objects are inside the device, as the window allows passage of visible light while inhibiting UV light from exiting the device.

The device 10 can include electronics, such as a main controller and one or more additional controllers in a distributed network architecture. The main controller may provide supervisory functions and management of operations and coordinate the performance of sub-operations by the other controllers. Each controller may include one or more processors that perform operations according to software that may be developed and compiled using one or more languages. The controllers may be in the form of embedded systems, having dedicated controllers, PLCs (programmable logic controllers), PC-based controllers with appropriate networking and I/O hardware and software, ASICs, or other implementations.

Again, with regard to FIG. 1B, the device 10 can include a treatment system 15 having the UV light source 22 and the control module 24. The control module 24 can include the microprocessor 26, memory 28, COM port 30, one or more sensors 32, one or more input/outputs 34 ports, power source 36, and the like. The various components of the control module 24 can be interconnected via the system bus 38.

The microprocessor 26 can be used to supervise, monitor, and control operations according to programmed instructions and/or hardware configurations (e.g., analog, digital, PAL, and/or ASIC circuits). The microprocessor 26 can be a single-threaded processor or a multi-threaded processor. The microprocessor 26 can be capable of processing instructions stored in the memory 28 and/or on a storage device to provide an output of information to the user about operation of the device 10. The microprocessor 26 can include software capable of being programmed. The software run by the microprocessor 26 can provide certain aspects of the device 10 without any user input during use. In an implementation, the adjustments or programming can be via the microprocessor 26 that is controlled by software.

The memory 28 may be part of the microprocessor 26 or otherwise in data communication with the microprocessor 26. The memory 28 is configured for receiving and storing user input data. The memory 28 can be configured to store user information, history of use, compliance monitoring, reminders, tracking, and the like. The memory 28 can be any type of memory capable of storing data and communicating that data to one or more other components of the device 10, such as the microprocessor 26. The memory 28 may be one or more of a Flash memory, SRAM, ROM, DRAM, RAM, EPROM, dynamic storage, and the like. The size of the memory 28 can vary as is known in the art.

The I/O port can be used to receive and send signals to the sensors 32 and/or other components in the device 10. The control module 24 may send and/or receive status and control information to or from a host computer or controller via the COM port 30. The input/outputs 34 may be combined or the input may be separate from the output. The one or more input/outputs 34 can include one or more buttons, sliders, dials, keypads, switches, touchscreens, or other input that can be retracted, pressed, squeezed, slid, tapped, or otherwise actuated to activate, modify, or otherwise cause a response of the device 10. The one or more input/outputs 34 can be optical (LED, display), tactile (e.g., vibrational, etc.), sonic (e.g., speaker, etc.), or the like. The one or more input/outputs 34 can also be more elaborate, such as a GUI having an input. The type of visual output/display may include LCD displays, LED displays, plasma displays, OLED displays and the like. The output/display may also be an interactive or touch sensitive screen having an input device, such as a touch screen, a capacitance screen, a resistive screen or the like. The one or more input/outputs 34 can also include a vibratory motor, speaker, warning, alarm, alert, clock, timer, or other features. The device preferably includes a single input that activates irradiation on a user-controlled basis that provides optimal timing of UV irradiation.

The input/outputs 34 can include one or more indicator lights that provide, for example, information about status or power of the device 10 as well as state of use of the device 10. As an example, a first color light in a first state may indicate a first status of the power source 36. The first color light in a second state may indicate a second status of the power source 36. For example, a red blinking light might indicate the battery is below about 10% power and red solid light might indicate the battery is dead. The light color can indicate whether the device 10 is ready to be used (e.g., green light) or is already in use (e.g., white light). The light can be a multi-color LED that communicates the administration status or dosing to assist a user.

The microprocessor 26 can be in operative communication with one or more peripheral devices, such as one or more external computing devices. The external computing device can vary including, but not limited to, desktop computer, laptop computer, tablet computer, smartphone, or other device capable of communicating and receiving user input. The connection between the device 10 and a peripheral device can include a wired communication port, such as a RS232 connection, USB, Fire wire connections, proprietary connections, or any other suitable type of hard-wired connection configured to receive and/or send information. The communication can also include a wireless communication port such that information can be fed to/from the system via a wireless link. The circuitry of the microprocessor 26 may include circuitry for one or more wireless communication modes, including Bluetooth, Internet of Things (IoT) connectivity, nearfield communication (NFC), Wi-Fi, ultrasound, ZigBee communication protocols, radio frequency, infrared, RFID, etc. The wireless connection can also be any of a variety of proprietary wireless connection protocols.

A battery or other power source 36 is disposed within the device 10 for storage and delivery of electrical power sufficient to power the device. The device can be recharged by micro-USB or USB-C cables. The device can additionally or alternatively incorporate a power cable that is plugged directly into the wall or other portable energy source as a means of a power instead of a battery. FIG. 7C shows an implementation of the device 10 having a power cord 60 connected to a backside of the device 10. The power cord 60 is coupled at a right-angle plug that is recessed within a groove 62 along a backside of the housing 12 so that the device 10 can be positioned flush with a wall. The groove 62 can extend along the entire width of the backside so that the cord 60 can extend left or right relative to the housing per a user's preference.

As discussed above, the device 10 can incorporate one or more sensors 32. At least one sensor 32 can detect whether the lid 40 is open and/or closed relative to the base 42. For example, the sensor 32 can detect if the lid 40 is closed and engaged with the base 42 allowing for the irradiating UV light source 22 to be activated once the lid 40 is closed. The sensor 32 can cause an automatic shut-off of the UV light source 22 upon opening of the lid 40 relative to the base. Alternatively, the sensor 32 can detect if the lid 40 is open and not engaged with the base 42 preventing the irradiating UV light source 22 to be activated until the lid 40 is closed. The device 10 can additionally incorporate one or more sensors 32 that detect a distance between the surface of the eye care accessory 5 beings disinfected and the UV light source 22. The device may also incorporate a kill switch to prevent use of the device after a certain number of activations.

The device can incorporate a sensor that is an “internet-of-things” (IoT)-connected sensor with artificial intelligence (AI)-driven analytics. The IoT-connected sensor can help patients and/or doctors track any of a variety of actions involving the device 10, such as eyedrop adherence to prescribed regimens, safety in administration of drops, ordering medication refills in timely manner, battery life, use (or non-use) of an adaptor 54, or other aspects relevant to the device 10. As an example, when a patient administers a treatment, such as applying a drop from a bottle contained within the device 10, an NB-IoT antenna within the device 10 can remotely update a server to which a physician has access. As part of a physician workflow in the clinic, a list can be provided to the physician from the server of any non-adherent patients over the past 24 hours.

In operation, a user opens the lid 40 revealing the interior cavity 50 of the housing 12 of the device 10. A first accessory 5a, such as an eye dropper bottle or goniolens or another accessory, can be placed within a first treatment bay 52a and a second accessory 5b, such as the cap of the eye dropper bottle or another accessory, can be placed within a second treatment bay 52b. The lid 40 can be closed against the base 42 sealing the first and second accessories within the interior cavity 50. The indicator 59 of the device 10 illuminates a first color or off/on pattern indicating the device 10 is ready for use. The device 10 can be ready for use upon the eye care accessory being properly loaded, the relevant surfaces of the accessories 5 being within a threshold distance from the UV light source 22, the lid 40 being sealed against the base 42, the power source 36 having sufficient power to complete a full disinfection protocol, and the like. If one or more of these criteria are not satisfied, the indicator 59 of the device 10 can illuminate a second color or a second off/on pattern indicating the device 10 is not ready for use. If the device 10 is not ready for use, even if the user presses the start button input 34, the device 10 will not initiate a disinfection protocol until the condition is resolved. Once the condition is resolved, the user can press the start button input 34 to initiate a disinfection protocol. Upon pressing the start button input 34, the UV light source 22 is powered on and the indicator 59 can illuminate a third color or a third off/on pattern informing the user that disinfection protocol has begun. The accessories can be cleaned prior to placing the accessory within the housing 12, for example, by removing foreign material (e.g., soil, sweat, tears, etc.) with a moist cloth, dry cloth, alcohol swab, and the like.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference and are not intended to limit the use or orientation of the devices to a specific configuration described in the various implementations.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value. In aspects, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1. A treatment device configured for storing and disinfecting one or more eye care accessories, the treatment device comprising:
a housing comprising at least one treatment bay sized to accommodate and support an eye care accessory; and
a plurality of ultraviolet light sources disposed within the housing, the plurality of ultraviolet light sources having an orientation, number, and radiant flux sufficient for achieving disinfection of the eye care accessory within the treatment bay.

2. (canceled)

3. The treatment device of claim 1, wherein the eye care accessory is an eyedropper bottle, eye dropper bottle cap, contact lens, contact lens case, applanation tonometer prism tip, rebound tonometer probe, ophthalmic prism lens, gonioprism, condensing lens, or other contact ophthalmic lens.

4. (canceled)

5. The treatment device of claim 1, wherein the treatment bay comprises a lower surface near a lower end region of the treatment bay, a ceiling near an upper end region that is positioned over at least a portion of the lower surface, and at least one side wall.

6. The treatment device of claim 5, wherein a first light source of the plurality of ultraviolet light sources is arranged within a first bank located within the at least one side wall.

7. The treatment device of claim 6, wherein the first bank comprises the first light source and a second light source, the first light source near the upper end region of the treatment bay and the second light source near the lower end region of the treatment bay.

8. The treatment device of claim 5, wherein at least one light source of the plurality of ultraviolet light sources is located within the ceiling of the treatment bay.

9.-12. (canceled)

13. The treatment device of claim 1, wherein the housing comprises three treatment bays, each treatment bay sized to accommodate and support one eye care accessory.

14. The treatment device of claim 13, wherein each bay of the three treatment bays comprises banks of light sources of the plurality of light sources, each bank of light sources arranged to create multiple overlapping cones of ultraviolet light.

15. The treatment device of claim 1, wherein each light source of the plurality of ultraviolet light sources is a UV-C light source that is a UV-C light emitting diode (LED).

16. (canceled)

17. The treatment device of claim 15, wherein the UV-C LED is powered to achieve high-level disinfection of the eye care accessory within an exposure period, wherein high-level disinfection causes 6 log reduction in bacterial concentration and 4 log reduction in viral concentration.

18. (canceled)

19. The treatment device of claim 17, wherein the exposure period is 1 minute to 5 minutes, and wherein the exposure period is cycled at least one cycle per day up to 100 cycles per day.

20. (canceled)

21. The treatment device of claim 15, wherein the UV-C LED has a threshold energy delivery that is at least 160 mJ/cm².

22. The treatment device of claim 5, wherein a first light source of the plurality of ultraviolet light sources is located within the ceiling of the treatment bay and a second light source of the plurality of ultraviolet light sources is located within the at least one side wall.

23. The treatment device of claim 22, wherein a radiant flux of the first light source is greater than a radiant flux of the second light source.

24. The treatment device of claim 22, wherein the second light source located within the at least one side wall comprises an upper light emitting diode (LED) separated vertically from a lower light emitting diode (LED), wherein the upper LED creates an upper cone of light and the lower LED creates a lower cone of light, wherein the upper cone of light and lower cone of light overlap.

25. The treatment device of claim 24, wherein a distance between the lower surface and the ceiling ensures that, when the eye care accessory is positioned on the lower surface, a target surface of the eye care accessory is located within a threshold treatment zone of the first light source.

26. The treatment device of claim 25, wherein a distance between the lower surface and the second light source ensures that, when the eye care accessory is positioned on the lower surface, the target surface of the eye care accessory is located within at least one of the upper cone of light and the lower cone of light.

27. The treatment device of claim 25, wherein the threshold treatment zone of the first light source is at least 7 mm and no greater than about 60 mm from the first light source.

28. (canceled)

29. The treatment device of claim 25, wherein the at least one of the upper cone of light and the lower cone of light is an 80-degree cone that directly exposes the target surface to a threshold energy delivery that is at least 160 mJ/cm².

30. The treatment device of claim 1, further comprising a microprocessor and a power system, wherein the microprocessor is programmed to provide timing of disinfection, and wherein the microprocessor comprises a wireless circuitry, wherein the wireless circuitry comprises Wi-Fi, Zigbee, Bluetooth, or Internet of Things (IoT) connectivity.

31.-50. (canceled)
